(19) United States
(12) Patent Application Publication
(10) Pub. No.: US 2011/0256185 A1
(43) Pub. Date: Oct. 20, 2011

(54) REINFORCED TISSUE SHIELDS

(75) Inventors: Liu Yang, Toronto (CA); Valerio DiTizio, Toronto (CA)

(73) Assignee: COVALON TECHNOLOGIES INC.

(21) Appl. No.: 13/087,920

(22) Filed: Apr. 15, 2011

Related U.S. Application Data
(60) Provisional application No. 61/324,460, filed on Apr. 15, 2010.

(51) Int. Cl. A61K 9/00; A61K 33/38; A61F 13/00; A61P 17/02; A61P 31/00; A61K 31/722; A61P 27/02

(52) U.S. Cl. 424/400; 514/55; 424/618; 602/43

(57) ABSTRACT

Self-reinforced tissue shields are useful as ophthalmic shields, wound dressings, wound barriers, nerve repair, therapeutic drug delivery devices and the like. The self-reinforced tissue protective shields comprise gelatin, chitosan and reinforce and are made by a method comprising forming inter-molecular locking within a solution through electrostatic forces, eliminating the use of extra cross-linking methods, the solution mainly comprising natural existing polymers that are biodegradable and biocompatible.

### RELATED APPLICATION

This application claims priority from U.S. Provisional Application Ser. No. 61/324,460, filed Apr. 15, 2010, the subject matter of which is herein incorporated by reference in its entirety.

### FIELD OF THE INVENTION

The present invention relates to protective tissue shields. More specifically, the invention relates to tissue shields for covering wounds, lesions or the like with a non-cross-linked wound shield that provides physical and therapeutic properties. The invention also relates to methods of making the non-cross-linked wound shield and methods of use.

### BACKGROUND OF THE INVENTION

There is an increasing need to manage complex wounds both in and out of the hospital. The highest incidence of wound infection usually occurs between 1 and 3 days after damage to the skin, an operation or trauma to skin, bone or other tissue. In particular, the incidence of ocular infection, i.e. endophthalmitis, is growing as a result of the rising number of ocular surgeries being performed, the widespread adoption of sutureless surgical techniques and a significant increase in the number of intravitreal injections. To reduce the occurrence of infection from ocular procedures a protective device is used post-operatively to protect the eye tissue from infection, and foster the growth of epithelial cells. Such ophthalmic shields are worn for several days and offer lubrication for the eye as well as a favorable environment that fosters the healing process.

Collagen has been typically used for optical shields for several years. Such collagen-based shields are shaped like contact lenses and are supplied in a dehydrated form, requiring rehydration prior to insertion. However, lens duration and shape preservation before dissolution is dictated by physically or chemically cross-linking the collagen. The predominant chemical agents that have been investigated for cross-linking collagen include chromium tanning, formaldehyde, glutaraldehyde, polyepoxy compounds, acyl azide, hexamethylenediisocyanate, and carbodiimides. Chemical methods typically utilize bi*functional chemicals that interact with collagen molecules at two different sites. The functional groups of the chemical agent react with those on the amino acid, such as the s-amino function on lysine and hydroxylysine or the carboxyl function on aspartic and glutamic acids to give rise to cross-links between the molecules. A drawback of chemical agents is the potential toxic effects a recipient may be exposed to from residues and/or chemicals resulting from a reversal of the cross-links (Kim et al, “Chitosan/Gelatin—Based Films Crosslinked by Proanthocyanidin”, J Biomed Mater Res Part B: Appl Biomater, 75B: 442-450 (2005)).

Physical cross-linking methods for collagen and gelatin include microwave energy, dehydrothermal treatment, and UV-irradiation. Unlike chemical cross-linking, physical methods do not introduce toxic chemicals into tissue, but this does not preclude undefined side-effects that may arise due to these processes such as significant shrinkage, limited degree of cross-linking, partial denaturation, molecular fragmentation, and reduced surface wettability. In addition, the efficiency and extent of these physical reactions depend on the thickness of the gelatin layers or solution, which defines the magnitude of penetration, the time and the temperature of exposure (Friess et al, “Collagen—biomaterial for drug delivery”, European Journal of Pharmaceutics and Biopharmaceutics, 45: 113-136 (1998)).

It is therefore desirable to provide a natural polymer-based tissue shield that is stable in a biological environment yet can eventually biodegrade over time. It is also desirable that such shield maintain its constructed shape without the use of extra cross-linking procedures or coatings.

### SUMMARY OF THE INVENTION

According to an aspect of the present invention there is a novel reinforced tissue shield comprising a blend of gelatin, chitosan and reinforcer. In an aspect, the tissue shield may be used to cover and/or protect a wide variety of tissues to inhibit microbial contamination. In an aspect, the tissue shield is a wound shield.

According to another aspect of the present invention there is a reinforced tissue shield comprising gelatin, chitosan, and a reinforcer, wherein the tissue shield is biocompatible and biodegradable in biological environments.

In an aspect, the tissue shield of claim 1, wherein the reinforcer maintains a desired shape of the tissue shield. In another aspect, the reinforcer is water swellable, has a fibrous structure, and provides mechanical strength.

According to an aspect, the reinforcer is insoluble collagen fibers, synthetic polymers, or combinations thereof. The reinforcer may be insoluble collagen fibers and may be present in an amount of from about 3 to about 40% or from about 3.5 to about 39% by weight of the total dry weight of the tissue shield. It is understood that the amount can be any range thereinbetween, in aspects 3 to about 8% by weight of the total dry weights of the tissue shield. The synthetic polymers may be in some aspects polyHEMA (poly-2-hydroxyethyl methacrylate), PMMA (polymethyl methacrylate) or combinations thereof.

In an aspect, the tissue shield is not cross-linked and the gelatin and chitosan may form inter-molecular complexes through electrostatic interaction. The tissue shield may have a desired dissolution rate that may be determined by pre-selecting a ratio of gelatin to chitosan in the tissue shield, wherein an increased ratio of gelatin to chitosan correlates to an increased dissolution rate.

According to an aspect, gelatin may be present in the tissue shield in an amount of from about 5 to about 70% or from about 6 to about 65% by weight of the total dry weight of the tissue shield. Sub-ranges of these ranges are encompassed, in aspects about 25 to about 70% and about 40 to about 65% by weight of the total dry weight of the tissue shield. Chitosan may be present in an amount of from about 5 to about 70% or from about 6 to about 65% by weight of the total dry weight of the tissue shield and the invention encompasses any desired sub-ranges thereof as is understood by one of skill in the art. In aspects this may be but is not limited to about 12 to about 57% and about 17 to about 42% by weight of the total dry weight of the tissue shield. The tissue shield may further comprise one or more of an additive and a pharmaceutical agent. In an aspect, the pharmaceutical agent is an antiseptic, an antimicrobial agent, an antibiotic, an antiproliferative agent, or combinations thereof. The antiseptic may be a silver salt, iodine, chlorhexidine, or combinations thereof. The silver salt may be silver nitrate, silver acetate, silver lactate, or combinations thereof. The silver salt can be used with or without the presence of their color stabilizer. The antibiotic may be selected from the group consisting of (but not limited to) aminoglycosides such as amikacin, gentamicin, kanamycin, tobramycin or a macrolide such as azithromycin, clarithromycin, erythromycin, or a β-lactam such as penicillin, piperacillin, cephalexin, cefazolin, cefixime, imipenem, or a quinolone such as nalidixic acid, ciprofloxacin, moxifloxacin, trovafloxacin, or clindamycin, vancomycin, rifampin, minocycline, or combinations thereof. The antiproliferative may be sirolimus.

According to another aspect, the tissue shield may comprise an additive, wherein the additive is a material that increases the flexibility and/or reduces the brittleness of the tissue shield. In an aspect, the additive may be polyethylene glycol (PEG), glycerol, or combinations thereof. The additive may be present in an amount of from about 5 to about 30% or in aspects from about 5 to about 15% by weight of the total dry weight of the tissue shield. Any sub-ranges of these ranges are encompassed by the present invention as is understood by one of skill in the art.

In an aspect of the invention, the tissue shield is hydrated or dehydrated and in another aspect, the tissue shield is a wound shield or an ophthalmic shield. The tissue shield may have a thickness of from about 0.02 mm to about 5 mm.

According to another aspect of the invention, there is a method of covering and/or protecting a wound, the method comprising applying a tissue shield as described herein to the wound. According to another aspect of the invention, there is a use of a tissue shield described herein for covering and/or protecting a wound. In an aspect, the tissue shield inhibits microbial contamination of the wound.

According to another aspect of the invention, there is a method of producing a reinforced tissue shield, the method comprising:
mixing gelatin, chitosan, and a reinforcer together to produce a matrix; and
dehydrating the matrix to produce the tissue shield.

According to an aspect, the tissue shield is formed into a desired shape without the use of chemical or physical cross-linking procedures. According to another aspect, the method further comprises molding the matrix into a desired shape prior to dehydrating the matrix. According to another aspect, the gelatin, chitosan, and reinforcer are produced as separate solutions that are then mixed together in a desired ratio to produce the matrix. The method may further comprise centrifuging the matrix to remove bubbles formed after mixing. In an aspect, the matrix is centrifuged for about 2 to about 30 minutes at a temperature of about 20° C. to about 30° C. at from about 1000 to about 3000 rpm.

According to another aspect, the method comprises dehydrating the matrix at about 20° C. to about 30° C. and from about 45 to about 80% relative humidity for about 48 hours or as desired as is understood by one of skill in the art. The method may further comprise placing the tissue shield in a vacuum oven for complete dehydration for about 4 to about 12 hours at a temperature of from about 23° C. to about 30° C. under a pressure from about 20 to about 30 mmHg According to another aspect, the dehydrated tissue shield is rehydrated prior to use. The dehydrated tissue shield may rehydrated in a solution comprising a therapeutically active agent. In another aspect, the dehydrated tissue shield may be rehydrated in a balanced physiological saline solution.

According to another aspect of the present invention, the method comprises transferring the liquid mixture of the gelatin, chitosan, and reinforcer comprising the matrix into an annular space between two differentially sized cylinders, degassing by centrifuging, and dehydrating the mixture through a freeze drying process over 24-36 hours for complete dehydration. According to this aspect, the dehydrated tissue shield is porous and has a tubular shape with a diameter ranging from about 1 to about 10 mm Such tubular and porous matrix may be used in nerve repair applications. According to an aspect of the present invention there is a novel reinforced tissue shield comprising a blend of gelatin, chitosan and reinforcer, wherein the reinforcer maintains a desired shape of the tissue shield.

According to an aspect of the present invention there is a novel reinforced tissue shield comprising a blend of gelatin, chitosan and reinforcer, wherein the reinforcer maintains a desired shape of the tissue shield and wherein said tissue shield is not cross-linked. In further aspects, the reinforced tissue shield does not require coatings or the like to maintain its shape.

According to another aspect of the present invention there is a novel reinforced tissue shield comprising a blend of gelatin, chitosan and reinforcer, wherein the reinforcer maintains a desired shape of the tissue shield, and wherein the gelatin and chitosan form inter-macromolecular complexes through electrostatic interaction.

In any of the aforementioned aspects, the reinforced tissue shield may further comprise one or more of an additive and a pharmaceutical agent. The reinforced tissue shield may be a reinforced wound shield or a reinforced ophthalmic protective shield.

According to another aspect of the invention there is a method for making a reinforced tissue shield, said method comprising:

(a) admixing solutions of gelatin and chitosan with a collagen dispersion,

(b) centrifuging (a); and

(c) subjecting (b) under suitable conditions of temperature, humidity and vacuum to form a dehydrated shield.

According to another aspect of the invention there is a method for making a reinforced tissue shield, said method consisting of:

(a) admixing solutions of gelatin and chitosan with a collagen dispersion,

(b) centrifuging (a);

(c) subjecting (b) under suitable conditions of temperature, humidity and vacuum to form a dehydrated shield; and

(d) optionally adding one or more of an additive and pharmaceutical agent.

According to another aspect of the invention, there is a dry reinforced tissue shield comprising gelatin, chitosan and reinforcer. In an aspect, the tissue shield is for covering and/or protecting wounds from microbial contamination and is referred to as a wound shield.

According to another aspect of the invention, there is a dry reinforced tissue shield comprising gelatin, chitosan and reinforcer, wherein said tissue shield is not cross-linked.

In aspects of the invention, the reinforced tissue shield has a thickness of about 0.02 mm to about 5 mm.

In further aspects of the invention, the reinforced tissue shield is flat or curved or tubular.

According to a further aspect of the present invention is a reinforced tissue shield with a desired dissolution rate.

According to another aspect of the present invention is a method for making a reinforced tissue shield with a desired shape without the use of either chemical or physical cross-linking procedures.

### BRIEF DESCRIPTION OF THE DRAWINGS

The present invention will become more fully understood from the detailed description given herein and from the accompanying drawings, which are given by way of illustration only and do not limit the intended scope of the invention.

FIG. 1 depicts a drawing of a mold that is used to make an ophthalmic protective shield with curvatures of both cornea and sclera, which can cover and conform to the shape of the eye;

FIG. 2 illustrates the influence of the ratio of gelatin to chitosan on the biodegradability of the tissue shield of the invention; and

FIG. 3 illustrates sustained silver release from a tissue shield of the invention.

### DETAILED DESCRIPTION OF THE EMBODIMENTS

The invention is directed to novel reinforced tissue shields and methods for making such shields. The novel reinforced tissue shields cover and/or protect a wide variety of tissues that require the use thereof, such as because of a wound or tissue damage that may require protective physical and/or therapeutic properties for inhibiting microbial contamination. The reinforced tissue shields may be used as, but not limited to, ophthalmic shields, wound dressings, wound barriers, for nerve repair/protection and in therapeutic drug, medicament and/or chemical agent delivery. In general, the reinforced tissue shields of the invention can be used in any medical wound application to reduce and/or inhibit potential microbial contamination.

The reinforced tissue shields of the invention comprise denatured collagen (gelatin), chitosan and a reinforcer such that the shield is reinforced. The gelatin and chitosan provide the major structural components and insoluble collagen is used in aspects as the reinforce.

As used herein, the term “reinforced” may be understood, in relative terms, that the stability of the tissue shield is achieved by utilizing the direct interaction between the materials, without using extra cross-linking procedures, so that the hydrated wound shields gradually dissolve, under physiological conditions, over a time period from one to a few days. The reinforced ability can be adjusted by varying the ratio of gelatin to chitosan and by employment of amounts of reinforcer. More generally, “reinforced” means that a desired shape (configuration) of the tissue shield of the invention is maintained.

The reinforced tissue shields of the present invention provide simplified preparation procedures (e.g. excluding the use of either physical or chemical cross-linking methods) for manufacturing shields, such as wound shields or ophthalmic shields, with controllable characteristics over the prior art collagen and/or gelatin-based versions. Specifically, the non-cross-linked gelatin and chitosan reinforced matrix from which the tissue shield is made provides a shield that is biocompatible and biodegradable in biological environments such as tear fluid. Moreover, the simplified procedure provides additional advantages over the physically or chemically cross-linked collagen and/or gelatin shields, including cost, ease of preparation, and freedom from adverse response related to toxic residual molecules and/or compounds formed during in vivo degradation.

Gelatin is a soluble protein obtained by breaking the triple-helix structure of collagen into single-strand molecules that possess the s-amino function on lysine and hydroxylysine and the carboxyl function on aspartic and glutamic acids. It is biodegradable, biocompatible, and essentially non-immunogenic, which makes it a suitable compound for biomedical applications. In aqueous solution, gelatin forms physical thermo-reversible gels upon lowering the temperature below 35° C. as the chains undergo a conformational coil-to-helix transition during which they tend to recover the collagen triple-helix structure. Gelatin for use in the present invention includes both Type A, derived from an acid-treated precursor, or Type B, derived from an alkali-treated precursor. Gelatin is advantageously employed in the tissue shields of the invention in concentrations ranging from about 5% to 70% by weight, based on the total weight of solids in the final dehydrated tissue shield product. In aspects of the invention, the gelatin concentrations are in the range from about 6% to about 65% by weight. However one of skill in the art would understand that any sub-ranges are contemplated by the present invention.

Chitosan is a biopolymer that is a naturally existing cationic polymer, partially deacetylated derivative of chitin. The N-acetylglucosamine in chitosan is a structural component also found in the glycosaminoglycans (GAGs). Chitosan has many useful and advantageous biological properties such as biocompatibility, biodegradability, hemostatic activity, anti-infective activity and the ability to accelerate wound-healing. It has been widely used for effective delivery of many pharmaceuticals. In addition, it also possesses characteristics required for suitable tissue shields, including ophthalmic shields, e.g. mechanical stability, gas permeability, wettability, and immunological compatibility (Dutta et al, Journal of Macromolecular Science; C42: 307-354, 2002). Chitosan is able to be broken down by lysozyme present at a relatively high and constant level, leading to acceptable dissolution properties of tissue and, more specifically, ophthalmic shields. In aspects of the invention, chitosan is in liquid form when mixed with gelatin and the reinforcer (which is, in aspects, insoluble collagen). Chitosan is used in the invention in concentrations ranging from about 5% to 70% by weight, based on the total weight of solids in the final product. In aspects suitable concentrations are in the range of from about 6% to about 65% by weight of the final dehydrated tissue shield.

Without being bound by any scientific theory, it is believed that inter-macromolecular complexes are formed between the molecules of gelatin and chitosan through the electrostatic interaction as these two materials carry ionized/ionizable groups of opposite charges under the specific formulation conditions used in this invention. The electrostatic forces may be completed by, for example, hydrogen bonds, van der Waals forces, and/or hydrophobic bonds.

Thus, the dissolution rate of the tissue shields can be adjusted by altering the ratio of gelatin to chitosan. In embodiments, the resultant reinforced tissue shield contains a ratio of gelatin to chitosan from about 0.2 to about 9 by weight, and in aspects, the ratio of gelatin to chitosan is in the range of from around 0.4 to around 4. This ratio of gelatin and chitosan may be varied to achieve different degrees of inter-molecular interlocking between these two materials through electrostatic interaction so as to provide an enhanced stability of gelatin and desired physical properties of the final product. Gelatin and chitosan provide the major (by weight %) structural components to the tissue shield of the invention, and the combination of these two ingredients at different ratios provides the unique structural and functional characteristics of the tissue shields. One such unique characteristic of the tissue shield of the invention is the ability to pre-select a desired dissolution rate.

To maintain a defined shape of the wound shield of the invention (such as concave shaped ophthalmic shields) over time, the reinforced tissue shield of the invention comprises a reinforcer. Suitable agents as reinforcers include any material that is water swellable, has a fibrous structure and provides mechanical strength. Examples of suitable reinforcers for use in the present invention include, for example, materials derived from insoluble fibrous polypeptides, polysaccharides, synthetic polymers, or combinations thereof. The insoluble fibrous polypeptides can be selected from keratins, fibroin, neurofilaments, vimentin-like proteins, lamins, collagen, or combinations thereof. The insoluble fibrous polysaccharides are selected from cellulose, alginate, pectin, chitin, or combinations thereof. The insoluble fibrous synthetic polymers are selected from polylactic acid, polycapralactone, polyurethane, polyacrylates such as polyHEMA (poly-2-hydroxyethyl methacrylate) and PMMA (polymethyl methacrylate) or combinations thereof.

In the present invention one or more reinforcers are added to the gelatin-chitosan matrix according to the present invention to produce a tissue shield. In this invention, insoluble fibrous collagen is advantageously used as a reinforcer to maintain a desired shape of the tissue protective shields, for instance, a concave semi-spherical shape, in physiological fluids with a purpose of eliminating the need for any cross-linking procedure. The collagen component may be easily incorporated by homogenously blending it with gelatin-chitosan during the manufacturing process. The insoluble collagen acts as a reinforcing component for improving the structural integrity of the tissue shields in the course of dissolution. The concentrations of insoluble collagen may be in the range of from about 3% to about 40% by weight. In aspects, the insoluble collagen will be present in an amount within the range of from about 3.5 to about 39% by weight of the final, dried, tissue shield. All sub-ranges thereof are contemplated as is understood by one of skill in the art. In other aspects of the invention, the self-reinforced wound protective shields may further include one or more therapeutically active substances (i.e. pharmaceutical agents) that will be released to the wounds upon dissolution of the shields. Therapeutically active substances can be incorporated into the inventive shields during the manufacturing process or during rehydration of the shields immediately prior to use. Any therapeutically active substance may be used in the tissue shields of the invention. In aspects the pharmaceutical agent is an antiseptic, an antimicrobial agent, an antibiotic, and anti-inflammatory, an antiproliferative agent, or a combination thereof. The antiseptic is selected from silver, iodine, phenols, terpenes, triarylmethane dyes, quaternary ammonium compounds such as chlorhexidine, polyhexamethylene biguanide, benzalkonium chloride, antimicrobial agents or combinations thereof. The silver is selected from silver nitrate, silver acetate, silver lactate, silver sulfate, silver citrate, silver phosphate, silver oxide, silver colloid, silver nanoparticles or combinations thereof. The antibiotic is selected from an aminoglycoside such as amikacin, gentamicin, kanamycin, tobramycin or a macrolide such as azithromycin, clarithromycin, erythromycin, or a B-lactam such as penicillin, piperacillin, cephalexin, cefazolin, cefixime, imipenem, or a quinolone such as nalidixic acid, ciprofloxacin, moxifloxacin, trovafloxacin, or clindamycin, vancomycin, rifampin, minocycline, or combinations thereof. The antiproliferative is selected from sirolimus, everolimus, halofuginone, paclitaxel, or combinations thereof. The anti-inflammatory is selected from a corticosteroid such as hydrocortisone, prednisolone, triamcinolone acetonide, dexamethasone, prednicarbate, or a non-steroidal anti-inflammatory such as aspirin, ibuprofen, naproxen, or combinations thereof.

The therapeutically active agents may be used alone or in combination in the tissue shields of the invention.

Other additive materials can be included in the tissue shields to give them desired flexible properties and/or to reduce brittleness. Examples of such materials are (but not limited to) polyethylene glycol (PEG), glycerol, propylene glycol, dibutyl phthalate, triacetin, triethyl citrate and the like as well any combinations thereof. Such materials may be incorporated in any amount that does not adversely affect the dissolution rate of the resultant shields or compromise the biocompatibility as is understood by one of skill in the art. The additional material is added in amounts of about 5% to about 30% of total solids weight of the dry tissue shield, but in aspects from about 5% to 15% by weight and any ranges therein between these.

If desired, other solids and/or other materials may also be incorporated into the shields of the invention, provided that these solids are biologically acceptable and provided that the total solids concentration is sufficient during the gradual dissolution of the shields. The concentration of the additional solids that may be useful is in the range of about 0.02% to about 2% by weight.

The shields of the invention may be prepared by suitable methods, including the three methods described herein below. In one method, the shields are produced by mixing all of the ingredients including the therapeutically active agent(s) together, and then the mixture is molded into the desired shield shape and type.

The methods for the manufacture of the wound shields depend on several parameters, such as biopolymer ratios and drying temperature and humidity. For example, to make contact lens-like ophthalmic shields, solutions of gelatin and chitosan as well as a dispersion of insoluble collagen can be prepared separately and mixed together at a desired ratio. Bubbles which may be formed during the mixing process can be removed by centrifuging the mixture for about 2 to about 30 minutes at a temperature of from about 20° C. to about 30° C. at from about 1000 to about 3000 rpm.

The mixture is then placed into a mold or tray of a desired shape. For example, when producing ophthalmic shields, concave semi-spherically shaped molds of single or multiple curvatures, usually made of polystyrene, polyethylene or polycarbonates. The mixture is then dried in an environment where temperature and humidity are controlled, for example, at about 20° C. to 30° C. and about 45 to 80% relative humidity for 48 hours to produce a tissue shield. Following the primary drying, the mold containing the shield then is placed in a vacuum oven for a complete drying for 4 to 12 hours at a temperature of from about 23° C. to about 30° C. under a pressure of from 20 to 30 mmHg Ophthalmic shields produced using this method and being shaped in a mold having a single curvature may be about 0.02 mm to about 0.06 mm thick with a radius of base curvature of about 9.0 mm or 12.25 mm and a diameter of about 14 mm or 20 mm, respectively, depending on the choice of the concave shaped molds (i.e. 9 mm radius and 14 mm diameter or 12 5 mm radius and 20 mm diameter). Sub-ranges may also be utilized.

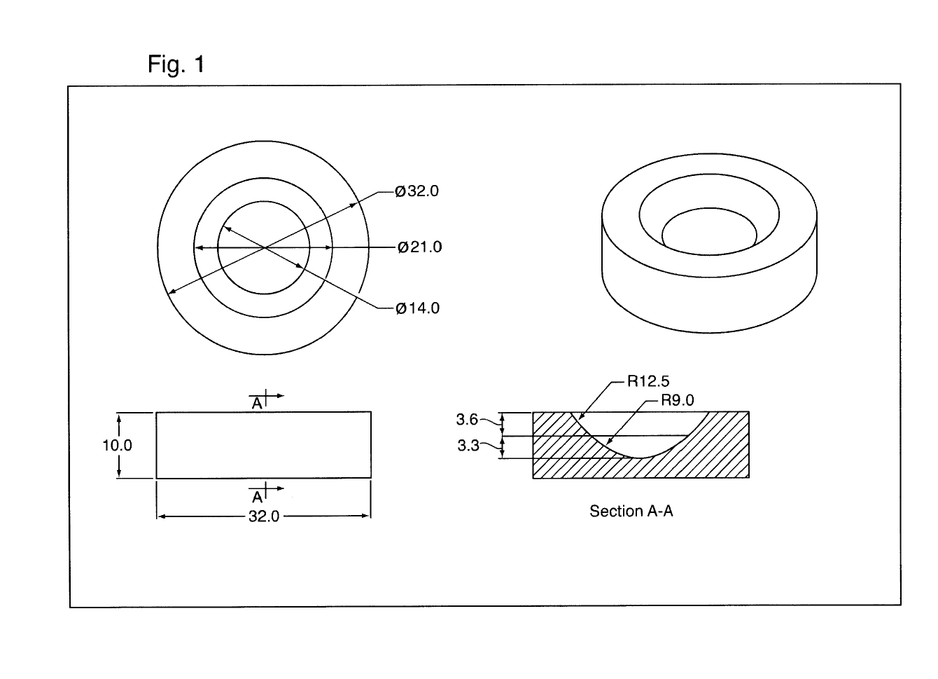

Ophthalmic shields can be also made by placing the mixture into molds having two radii of curvatures (FIG. 1). The resultant shield can be placed on the eye as a whole to cover the entire eye or made into a ring with the center removed (i.e. ring having an aperature therein of varying size as selected by one of skill in the art) using a sterile hole puncher to cover wounds only located in the sclera. For covering wounds located only in the corneal area, the center part of the shields of two radii can be punched and then applied onto the cornea.

Following manufacturing, the tissue shields may be stored in the dried condition at room temperature and supplied in a dehydrated form in sterile, individual packaging until they need to be used. For use following eye operations, the shields first must be rehydrated either in the sterile packaging or in a sterile receptacle with the therapeutically active agent of choice and/or with a balanced physiological saline solution for about ten minutes.

After placement of the shields on the eye, the eye may be patched or left unpatched, although patching the eye is preferred, at least for about the first twenty-four hours after application of a shield. The biodegradation of the shields by the enzymes in the eye offers the additional advantages of eliminating any need for removal of the shields once they have been applied on to the eye.

To make the tissue protective shields in other types of shapes, such as a film, the mixture may be poured into flat trays made of the above-mentioned materials and being of any size, such as 2×2 in., or 4×4 in. and dried in the same manner, resulting in flat sheets that are about 0.03 mm to about 0.35 mm thick. In another aspect of the invention, the shields cast as flat sheets may be cut into any shape using scissors as the clinical need dictates.

To make a tubular tissue protective shield, the mixture may be transferred into annular space between two cylinders made of silicone, polyurethane, polystyrene, polyethylene or polycarbonates and being of sizes such as but not limited to about 2 cm to about 10 cm long. The mixture may be dried using freeze drying process. For example, the mixture containing device may be frozen and lyophilized at about −35° C. for about 30 hours. The resulted porous and tubular matrix may have a thickness from about 3 mm to about 7 mm and a diameter from about 1 to about 10 mm Both depend on the diameters of the two cylinders. It may be cut into any length using scissors or any suitable cutter as the clinical need dictates.

The above disclosure generally describes the present invention. A more complete understanding can be obtained by reference to the following specific Examples. These Examples are described solely for purposes of illustration and are not intended to limit the scope of the invention. Changes in form and substitution of equivalents are contemplated as circumstances may suggest or render expedient. Although specific terms have been employed herein, such terms are intended in a descriptive sense and not for purposes of limitation.

### EXAMPLES

### Example 1

### Fabrication of Self-Reinforced Ophthalmic Protective Shields of Single Curvature

1.5% gelatin solution is prepared by dissolving gelatin powder in distilled water. 1.5% chitosan solution is prepared by dissolving chitosan of 85% deacetylation degree in 1% acetic acid water solution. 1% insoluble collagen is swelled and dispersed in 1% acetic acid water. The solutions of gelatin and chitosan and collagen dispersion are combined together along with addition of polyethylene glycol 400 (PEG400) and therein mixed for 2 hours to yield a dispersion having a total solids concentration of 1.7%, and such that on drying the solution yields a gelatin concentration of 42%, chitosan 42%, PEG400 12%, and insoluble collagen 4%. The combined dispersion is then centrifuged for 2 minutes at 3000 rpm at room temperature.

The dispersion is placed into concave semi-spherically shaped molds having a radius of base curvature of 12.5 mm and a diameter of 20 mm The dispersion is allowed to dehydrate in a vibration free environment under controlled temperature (25±3° C.) and humidity (55-80%) conditions. After approximately 48 hours of drying, the shields, still in the molds, are subjected to further drying in a vacuum oven for 4 hours. The second step in the drying process is carried out under an atmosphere of about 25 mm of mercury and at a temperature around 25±3° C.

The shields, in the form of concave-shaped thin membranes, are packaged within the molds. The sealed packages are sterilized using gamma irradiation. The resulting shields exhibit excellent stability and shape-maintaining ability in balanced physiological saline.

### Example 2

### Fabrication of Self-Reinforced Therapeutic Protective Shields

A gelatin solution is prepared by dissolving 1.5 gram of gelatin powder in 100 ml distilled water. 1.5% chitosan solution is prepared by dissolving chitosan of 85% deacetylation degree in 1% acetic acid water solution. Insoluble collagen suspension is prepared by blending 1 gram of insoluble fibrous collagen in 100 ml of 1% acetic acid water at 4° C. for 4 minutes. The solutions of gelatin and chitosan, collagen dispersion and PEG 400 are combined together and then mixed for 2 hours, followed by addition of silver acetate and sodium chloride to yield 5 mM silver chloride in the final suspension that has a total solid concentration of 1.8%. The combined dispersion is then centrifuged for 2 minutes at 3000 rpm at room temperature. 12 ml of the dispersion is placed in a 2″×2″ flat polystyrene tray and allowed to dehydrate in a vibration free environment under controlled temperature (25±3° C.) and humidity (55-80%) conditions. After approximately 48 hours, the dried solution, still in the tray, is subjected to a further drying in a vacuum oven for 4 hours, resulting in a grayish and pliable membrane having a gelatin concentration of 23%, chitosan 53%, PEG400 11%, insoluble collagen 6%, silver chloride 6% and other solids of 1%.

### Example 3

### Degradation of the Self-Reinforced Ophthalmic Shields (the Effect of Ratio of Gelatin To Chitosan On the Degradability)

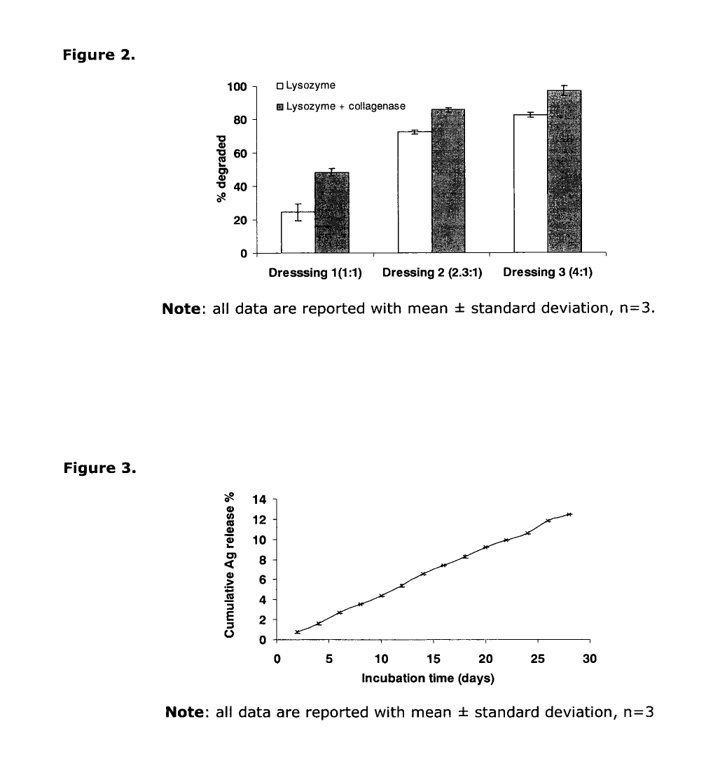

Three groups of concave shaped ophthalmic shields with ratios of gelatin to chitosan of 1:1, 2.3:1, and 4:1 are incubated in simulated tear fluid with 0.1% lysozyme with and without 12.5 unit/ml collagenase at 37° C. for 24 hours. The degradability of the shields is evaluated by comparing the dry weight of each shield before and after the incubation. Shields with the highest ratio of gelatin to chitosan (4:1) were almost completely dissolved in a simulated tear fluid (STF) containing both lysozyme and collagenase. The degradability of these shields in lysozyme-containing STF with or without collagenase is significantly influenced by the ratio of gelatin to chitosan (FIG. 2, p<0.05). Exemplary suitable polymer concentrations for formulating a 24 hr ophthalmic shield are 67% gelatin, 17% chitosan, 12% PEG400, and 4% insoluble collagen. For a 72 hr shield, a composition of, for example, 42% gelatin and 42% chitosan is suitable.

### Example 4

### Silver Release From the Protective Membranes

Sustained release of silver from the protective membranes is particularly important and applicable when employing such membranes as protective barriers to prevent bacterial infection. Each 4 cm2 membrane is incubated in 12 mL of phosphate buffered saline (PBS; pH 7.2) at 37° C. and transferred to the equivalent amount of fresh PBS medium every other day until 28 days. The Ag content in the collected PBS solution is analyzed immediately using an atomic absorption spectrometer (Varian SpectrAA-50). FIG. 3 shows a constant and slow silver release, around 0.9% every 2 days, and demonstrates a linear and sustained Ag release behavior (R²=0.9982) over the 28 days of incubation. These results clearly demonstrate the ability of these non-cross-linked membranes serving as suitable matrix for gradual and stable delivery of antimicrobial Ag.

### Example 5

### Antimicrobial Activity of the Protective Membranes For Use In Podiatric Ulcer Treatment

Gelatin and chitosan solutions and insoluble collagen suspension were separately prepared, combined, mixed and centrifuged as described in Example 2. Silver acetate was dissolved in a small quantity of water first and added into the dispersion followed by addition of chlorhexidine digluconate aqueous solution (i.e. commercially available 20% CHG aqueous solution) to yield 1.4 mM silver acetate and 5.5 mM chlorhexidine digluconate in the final dispersion that has a total solid concentration of 1% and 28%, respectively. The dispersion was dried following the procedure described in Example 2. The resulting film was pliable membrane having a gelatin concentration of 13%, chitosan 13%, PEG400 7%, insoluble collagen 38%, silver acetate 1% and chlorhexidine 28%.

Kirby-Bauer test (zone of inhibition) was performed to examine the antimicrobial activity against S. aureus by placing the membranes on a Mueller-Hinton agar surface inoculated with S. aureus (ATCC 6538) and incubated at 35±2° C. The zone of inhibition refers to the clear area around the sample that is free of microorganisms. It is calculated by subtracting the width of the sample from the width (across the center of the square sample) of the clear area including the sample. The zone of inhibition is expressed in millimeters. Neat membranes (films without Ag and CHG) were used as a control in this study for comparison purposes. Antimicrobial activity in terms of zone of inhibition lasted over 19 days.

### Example 6

### Color Stable Protective Membranes

Gelatin and chitosan solutions and insoluble collagen suspension were separately prepared, combined, mixed and centrifuged as described in Example 2. Silver acetate was dissolved in a small quantity of water first and added into the dispersion followed by addition of Pyroglutamic acid that was also pre-dissolved in a small quantity of water to yield a 5 mM silver acetate and 5 mM pyroglutamic acid in final dispersion that has a final solid concentration of 4.9% and 3.8%, respectively. The final dispersion was dried following the procedure described in Example 2. The resulting film was pliable membrane having a gelatin concentration of 46.1%, chitosan 27.6%, PEG400 7.7%, insoluble collagen 9.8%, silver acetate 4.9% and pyroglutamic acid 3.8%. A comparative membrane was also made in the same manner in the presence of pyroglutamic acid for comparison purpose. The films were placed on bench and exposed to normal lighting for 7 days in order to examine the color change. The membrane containing silver acetate and pyroglutamic acid maintained its clarity and color stability over 7 days while its counterpart that did not have pyroglutamic acid turned dark grey after 1 day.

1. A reinforced tissue shield comprising gelatin, chitosan, and a reinforcer, wherein the tissue shield is biocompatible and biodegradable in biological environments.

2. The tissue shield of claim 1, wherein the reinforcer maintains a desired shape of the tissue shield.

3. The tissue shield of claim 2, wherein the reinforcer is water swellable, has a fibrous structure, and provides mechanical strength.

4. The tissue shield of claim 1, wherein the reinforcer is selected from insoluble fibrous polypeptides, polysaccharides, synthetic polymers and combinations thereof.

5. The tissue shield of claim 4, wherein the insoluble fibrous polypeptides are selected from the group consisting of keratins, fibrin, neurofilaments, vimentin-like proteins, lamins, collagen and combinations thereof.

6. The tissue shield of claim 4, wherein the insoluble fibrous polysaccharides are selected from the group consisting cellulose, alginate, pectin, chitin and combinations thereof.

7. The tissue shield of claim 4, wherein the insoluble fibrous synthetic polymers are selected from the group consisting of polylactic acid, polycapralactone, polyurethane, polyacrylates selected from polyHEMA and PMMA and combinations thereof.

8. The tissue shield of claim 5, wherein the reinforcer is insoluble collagen fibers present in an amount of from about 3 to about 8% by weight of the total dry weight of the tissue shield.

9. The tissue shield of claim 5, wherein the reinforcer is insoluble collagen fibers and is present in an amount of from about 3.5 to about 39% by weight of the total dry weight of the tissue shield.

10. The tissue shield of claim 1, wherein the tissue shield is not cross-linked.

11. The tissue shield of claim 1, wherein the gelatin and chitosan form inter-molecular complexes through electrostatic interaction.

12. The tissue shield of claim 1, wherein the tissue shield has a desired dissolution rate.

13. The tissue shield of claim 1, wherein the desired dissolution rate is determined by pre-selecting a ratio of gelatin to chitosan in the tissue shield, wherein an increased ratio of gelatin to chitosan correlates to an increased dissolution rate.

14. The tissue shield of claim 1, wherein the gelatin is present in an amount of from about 5 to about 70% by weight of the total dry weight of the tissue shield.

15. The tissue shield of claim 14, wherein the gelatin is present in an amount of from about 6 to about 65% by weight of the total dry weight of the tissue shield.

16. The tissue shield of claim 1, wherein the chitosan is present in an amount of from about 5 to about 70% by weight of the total dry weight of the tissue shield.

17. The tissue shield of claim 1, wherein the chitosan is present in an amount of from about 6 to about 65% by weight of the total dry weight of the tissue shield.

18. The tissue shield of claim 1, further comprising one or more of an additive and a pharmaceutical agent.

19. The tissue shield of claim 16, wherein the pharmaceutical agent is an antiseptic, an antimicrobial agent, an antibiotic, and anti-inflammatory, an antiproliferative agent, or combinations thereof.

20. The tissue shield of claim 19, wherein the antiseptic is silver, iodine, phenols, terpenes, triarylmethane dyes, quaternary ammonium compounds selected from chlorhexidine, polyhexamethylene biguanide, benzalkonium chloride, and combinations thereof.

21. The tissue shield of claim 20, wherein the silver is silver nitrate, silver acetate, silver lactate, silver sulfate, silver citrate, silver phosphate, silver oxide, silver colloid, silver nanoparticles or combinations thereof.

22. The tissue shield of claim 19, wherein the antibiotic is an aminoglycoside selected from amikacin, gentamicin, kanamycin, tobramycin or a macrolide selected from azithromycin, clarithromycin, erythromycin, or a B-lactam selected from penicillin, piperacillin, cephalexin, cefazolin, cefixime, imipenem, or a quinolone selected from nalidixic acid, ciprofloxacin, moxifloxacin, trovafloxacin, or clindamycin, vancomycin, rifampin, minocycline, and combinations thereof.

23. The tissue shield of claim 19, wherein the antiproliferative is selected from sirolimus, everolimus, halofuginone, paclitaxel, and combinations thereof.

24. The tissue shield of claim 19, wherein the anti-inflammatory is a corticosteroid selected from hydrocortisone, prednisolone, triamcinolone acetonide, dexamethasone, prednicarbate, or a non-steroidal anti-inflammatory selected from aspirin, ibuprofen, naproxen, and combinations thereof.

25. The tissue shield of claim 18, wherein the additive is a material that increases the flexibility and/or reduces the brittleness of the tissue shield.

26. The tissue shield of claim 25, wherein the additive is selected from polyethylene glycol (PEG), glycerol, propylene glycol, dibutyl phthalate, triacetin, triethyl citrate, and combinations thereof.

27. The tissue shield of claim 25, wherein the additive is present in an amount of from about 5 to about 30% by weight of the total dry weight of the tissue shield.

28. The tissue shield of claim 27, wherein the additive is present in an amount of from about 5 to about 15% by weight of the total dry weight of the tissue shield.

29. The tissue shield of claim 1, wherein the tissue shield is flat, tubular or curved.

30. The tissue shield of claim 1, wherein the tissue shield is hydrated or dehydrated.

31. The tissue shield of claim 1, wherein the tissue shield is a wound shield.

32. The tissue shield of claim 1, wherein the tissue shield has a thickness of from about 0.02 mm to about 0.35 mm.

33. The tissue shield of claim 1, wherein the tissue shield is an ophthalmic shield.

34. The tissue shield of claim 33, comprising a single curvature of about 0.02 to about 0.06 mm thickness and a radius of base curvature of about 9.0 to about 12.25 mm and a diameter of about 14 mm to about 20 mm.

35. The tissue shield of claim 33, wherein said shield is configured as a curved disc or a ring having an aperature therein.

36. A method of covering and/or protecting a wound, the method comprising applying the tissue shield of claim 1 to the wound.

37. The method of claim 36, wherein the tissue shield inhibits microbial contamination of the wound.

38. A method of producing a reinforced tissue shield, the method comprising:
mixing gelatin, chitosan, and a reinforcer together to produce a matrix; and
dehydrating the matrix to produce the tissue shield.

39. The method of claim 38, wherein the tissue shield is formed into a desired shape without the use of chemical or physical cross-linking procedures.

40. The method of claim 38, further comprising molding the matrix into a desired shape prior to dehydrating the matrix.

41. The method of claim 38, wherein the gelatin, chitosan, and reinforcer are produced as separate solutions that are then mixed together in a desired ratio to produce the matrix.

42. The method of claim 38, further comprising centrifuging the matrix to remove bubbles formed after mixing.

43. The method of claim 42, wherein the matrix is centrifuged for about 2 to about 30 minutes at a temperature of about 20° C. to about 30° C. at from about 1000 to about 3000 rpm.

44. The method of claim 38 further comprising dehydrating the matrix at about 20° C. to about 30° C. and from about 45 to about 80% relative humidity for about 48 hours.

45. The method of claim 44, further comprising placing the tissue shield in a vacuum oven for complete dehydration for about 4 to about 12 hours at a temperature of from about 23° C. to about 30° C. under a pressure from about 20 to about 30 mmHg.

46. The method of claim 38, wherein the dehydrated tissue shield is rehydrated prior to use.

47. The method of claim 46, wherein the dehydrated tissue shield is rehydrated in a solution comprising a therapeutically active agent.

48. The method of claim 46, wherein the dehydrated tissue shield is rehydrated in a balanced physiological saline solution.
